(19) United States
(12) Patent Application Publication
(10) Pub. No.: US 2026/0212881 A1
(43) Pub. Date: Jul. 23, 2026

(54) MONITORING SPEECH-LANGUAGE MILESTONES

(71) Applicant: Cochlear Limited, Macquarie University, NSW (AU)

(72) Inventors: Paul REINHART, Alexandria, VA (US); Bridget TIERNAN, Lone Tree, CO (US)

(21) Appl. No.: 19/140,138

(22) PCT Filed: Jan. 5, 2024
(86) PCT No.: PCT/IB2024/050108

§ 371 Date: Jun. 17, 2025

Related U.S. Application Data
(60) Provisional application No. 63/479,617, filed on Jan. 12, 2023.

(51) Int. Cl. G10L 25/66; A61B 5/00; A61B 5/11; G10L 25/84

(52) U.S. Cl. CPC G10L 25/66; A61B 5/1114; A61B 5/4803; G10L 25/84

(57) ABSTRACT

Presented herein are techniques for detecting, tracking, and/or monitoring (collectively and generally “monitoring”) speech-language milestones of a user (e.g., recipient) of a “user device,” that is a device that is carried by, worn by, or implanted in, the user. The speech-language milestones can include, but are not limited, to pediatric speech-language development milestones.

### BACKGROUND

### Field of the Invention

The present invention relates generally to monitoring speech-language milestones of a user.

### Related Art

Medical devices have provided a wide range of therapeutic benefits to recipients over recent decades. Medical devices can include internal or implantable components/devices, external or wearable components/devices, or combinations thereof (e.g., a device having an external component communicating with an implantable component). Medical devices, such as traditional hearing aids, partially or fully-implantable hearing prostheses (e.g., bone conduction devices, mechanical stimulators, cochlear implants, etc.), pacemakers, defibrillators, functional electrical stimulation devices, and other medical devices, have been successful in performing lifesaving and/or lifestyle enhancement functions and/or recipient monitoring for a number of years.

The types of medical devices and the ranges of functions performed thereby have increased over the years. For example, many medical devices, sometimes referred to as “implantable medical devices,” now often include one or more instruments, apparatus, sensors, processors, controllers or other functional mechanical or electrical components that are permanently or temporarily implanted in a recipient. These functional devices are typically used to diagnose, prevent, monitor, treat, or manage a disease/injury or symptom thereof, or to investigate, replace or modify the anatomy or a physiological process. Many of these functional devices utilize power and/or data received from external devices that are part of, or operate in conjunction with, implantable components.

### SUMMARY

In one aspect, a method is provided. The method comprises: obtaining motion data from at least one motion sensor in a hearing device configured to be worn at a head of a user; and determining, based at least in part on the motion data, whether the user is meeting one or more predetermined speech-language milestones.

In another aspect, one or more non-transitory computer readable storage media are provided. The one or more non-transitory computer readable storage media comprise instructions that, when executed by a processor, cause the processor to: obtain acoustic data from at least one acoustic detector in a user device associated with a user; and determine, based at least in part on the acoustic data, whether the user is meeting one or more predetermined speech-language milestones.

In another aspect, another method is provided. The method comprises: capturing, by at least one acoustic detector in a hearing device configured to be worn at a head of a user, acoustic data associated with the user; and determining whether the user is reaching one or more predetermined speech-language milestones based on the acoustic data.

In another aspect, a device is provided. The device comprises: a network adapter for communication with a user device associated with a user, where the user device includes one or more motion sensors and one or more one or more acoustic detectors; memory; one or more processors, wherein the one or more processors are configured to: obtain motion data associated with the user from the one or more motion sensors, obtain acoustic data from the one or more acoustic detectors, determine whether the user of the user device is meeting one or more predetermined speech-language milestones based on at least one of the motion data or the acoustic data, and one or more output devices configured to provide an indication of whether the user of the user device is meeting the one or more predetermined speech-language milestones.

### BRIEF DESCRIPTION OF THE DRAWINGS

Embodiments of the present invention are described herein in conjunction with the accompanying drawings, in which:

FIG. 1A is a schematic diagram illustrating a cochlear implant system with which aspects of the techniques presented herein can be implemented;

FIG. 1B is a side view of a user wearing a sound processing unit of the cochlear implant system of FIG. 1A;

FIG. 1C is a schematic view of components of the cochlear implant system of FIG. 1A;

FIG. 1D is a block diagram of the cochlear implant system of FIG. 1A;

FIG. 1E is a schematic diagram illustrating a computing device with which aspects of the techniques presented herein can be implemented;

FIG. 2 is a table illustrating various example speech-language development milestones and accompanying data for use in assessing pediatric speech-language development, in accordance with certain embodiments presented herein;

FIG. 3 is a table illustrating various common calculated speech-language patterns for use in assessing pediatric speech-language development, in accordance with certain embodiments presented herein;

FIG. 4A is a schematic diagram illustrating assessment of a child's speech-language development based on age-specific normative values, in accordance with certain embodiments presented herein;

FIG. 4B is a schematic diagram illustrating use of acoustic data to detect and track appropriate turn-taking and speech sound imitation, in accordance with certain embodiments presented herein;

FIG. 4C is a schematic diagram illustrating use of acoustic data to detect and monitor novel word production, and to track use of new words and vocabulary growth over time, in accordance with certain embodiments presented herein;

FIG. 4D is a schematic diagram illustrating use of acoustic data to monitor speech fluency and detect dysfluencies atypical of a child's age, in accordance with certain embodiments presented herein;

FIG. 5A is a flowchart illustrating a method for identifying pediatric speech-language development milestones based on sensor data and/or acoustic data, in accordance with certain embodiments presented herein;

FIG. 5B is a flowchart illustrating another method for identifying pediatric speech-language development milestones based on sensor data and/or acoustic data, in accordance with certain embodiments presented herein;

FIG. 5C is a flowchart illustrating a method for detecting speech-language changes in adults as potential predictors of cognitive decline or speech production decline based on acoustic data, in accordance with certain embodiments presented herein;

FIG. 6 is a functional block diagram of an implantable stimulator system with which aspects of the techniques presented herein can be implemented;

FIG. 7 is a schematic diagram illustrating a vestibular nerve stimulator system with which aspects of the techniques presented herein can be implemented;

FIG. 8 is a flowchart of an example method, in accordance with certain embodiments presented herein;

FIG. 9 is a flowchart of an example method, in accordance with certain embodiments presented herein; and

FIG. 10 is a flowchart of an example method, in accordance with certain embodiments presented herein.

### DETAILED DESCRIPTION

Presented herein are techniques for detecting, tracking, and/or monitoring (collectively and generally “monitoring”) speech-language milestones of a user (e.g., recipient) of a “user device,” that is a device that is carried by, worn by, or implanted in, the user. The speech-language milestones can include, but are not limited, to pediatric speech-language development milestones.

As described in greater detail below, a user device (e.g., a portable device carried by a user, a wearable device worn by a user, or an implantable device having one or more components implanted in the user) is configured to capture data that can be used to monitor the speech-language milestones of the device user. The captured data can include, for example, sensor data (e.g., motion data captured by sensors of the user device) and/or acoustic data associated with a user. For example, when the user device is embodied as a hearing device, such as a cochlear implant or hearing aid, a speech-language milestone of a user of the hearing device can be identified based on sensor data (e.g., motion data) associated with the user. In addition, a microphone or acoustic detector of the hearing device can be used to identify the speech-language milestones and/or distinguish between different milestones.

In certain examples, the techniques presented herein can be beneficial for identifying speech-language developmental milestones of pediatric users of hearing devices and tracking/monitoring whether the pediatric users are on target to reach developmental milestones. Parents and pediatricians often find it useful to track speech-language developmental milestones to determine if a child is meeting the age-appropriate targets for language skills. If it is detected that a child is not meeting speech-language developmental milestones, additional diagnoses or assistance can be provided. In addition, in cases of children with hearing loss, being able to communicate to parents/caregivers and clinicians whether or not a hearing-impaired child is meeting his or her speech-language developmental milestones could give peace of mind to the parents/caregivers. Additionally, if a child is not meeting speech-language developmental milestones, technological interventions or rehabilitations could potentially be prescribed at an early age. However, example embodiments are not limited to pediatric users, and in certain other examples, some of the techniques presented herein can be beneficial for monitoring speech-language milestones (e.g., associated with cognitive decline and/or speech production decline) for adult users.

There are a number of different types of user devices in/with which embodiments of the present invention can be implemented. Merely for ease of description and illustration, the techniques presented herein are primarily described with reference to a specific user device in the form of a cochlear implant system. However, it is to be appreciated that the techniques presented herein can also be partially or fully implemented by any of a number of different types of user devices, including consumer electronic device (e.g., mobile phones), wearable devices (e.g., smartwatches), hearing devices, implantable medical devices, wearable devices, etc. consumer electronic devices, wearable devices (e.g., smart watches, etc.), etc. As used herein, the term “hearing device” is to be broadly construed as any device that delivers sound signals to a user in any form, including in the form of acoustical stimulation, mechanical stimulation, electrical stimulation, etc. As such, a hearing device can be a device for use by a hearing-impaired person (e.g., hearing aids, middle ear auditory prostheses, bone conduction devices, direct acoustic stimulators, electro-acoustic hearing prostheses, auditory brainstem stimulators, bimodal hearing prostheses, bilateral hearing prostheses, dedicated tinnitus therapy devices, tinnitus therapy device systems, combinations or variations thereof, etc.) or a device for use by a person with normal hearing (e.g., consumer devices that provide audio streaming, consumer headphones, earphones and other listening devices). In other examples, the techniques presented herein can be implemented by, or used in conjunction with, various implantable medical devices, such as vestibular devices (e.g., vestibular implants), visual devices (i.e., bionic eyes), sensors, pacemakers, drug delivery systems, defibrillators, functional electrical stimulation devices, catheters, seizure devices (e.g., devices for monitoring and/or treating epileptic events), sleep apnea devices, electroporation devices, etc.

### Example System

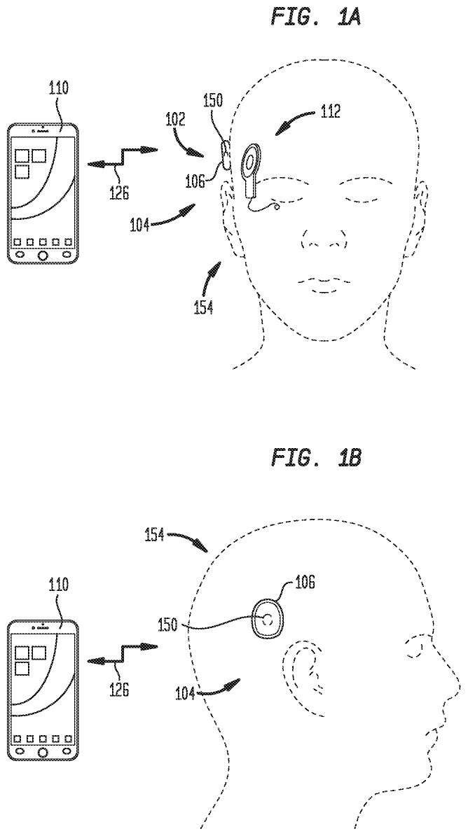

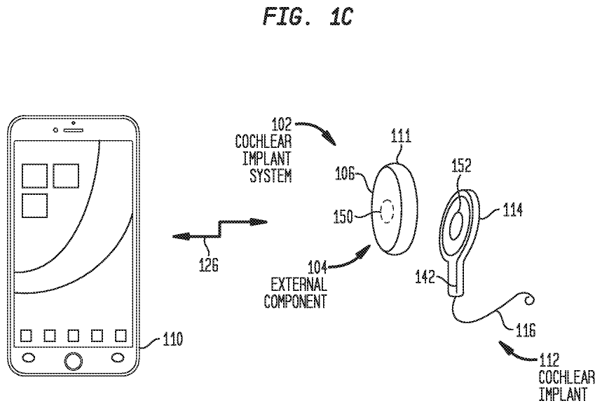

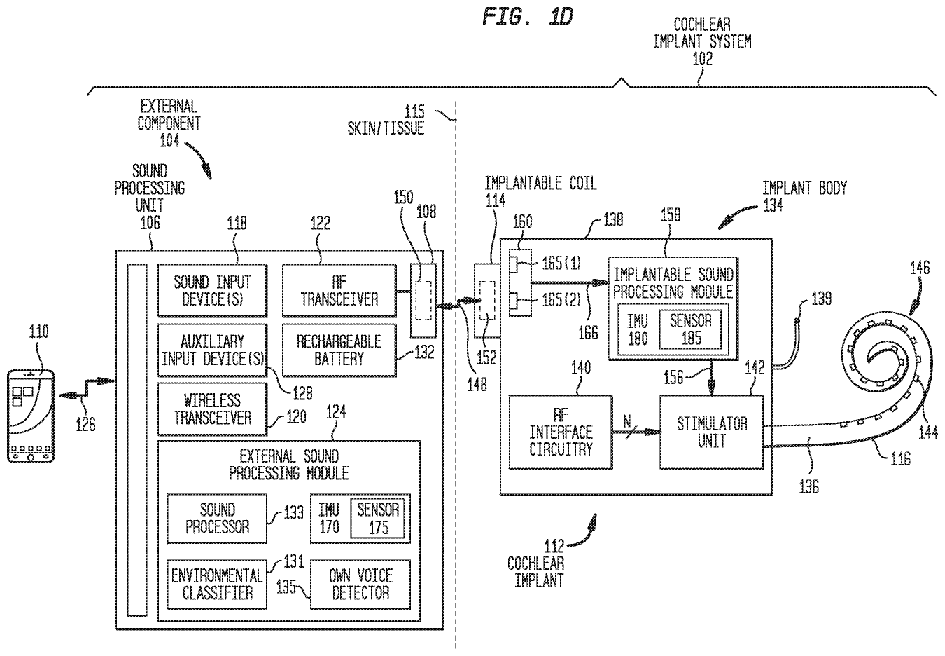

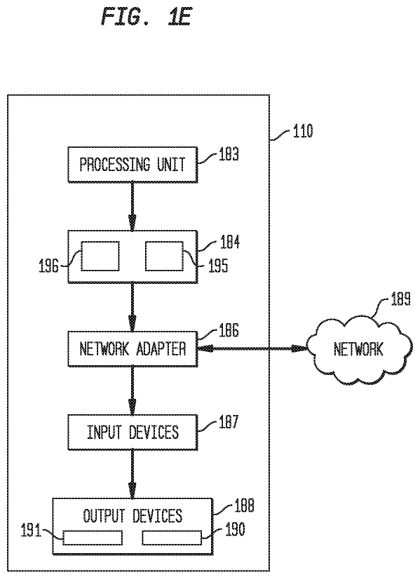

FIGS. 1A-1D illustrates an example cochlear implant system 102 with which aspects of the techniques presented herein can be implemented. The cochlear implant system 102 comprises an external component 104 that is configured to be directly or indirectly attached to the body of the user, and an internal/implantable component 112 that is configured to be implanted in or worn on the head of the user. In the examples of FIGS. 1A-1D, the implantable component 112 is sometimes referred to as a “cochlear implant.” FIG. 1A illustrates the cochlear implant 112 implanted in the head 154 of a user, while FIG. 1B is a schematic drawing of the external component 104 worn on the head 154 of the user. FIG. 1C is another schematic view of the cochlear implant system 102, while FIG. 1D illustrates further details of the cochlear implant system 102. For ease of description, FIGS. 1A-1D will generally be described together.

In the examples of FIGS. 1A-1D, the external component 104 comprises a sound processing unit 106, an external coil 108, and generally, a magnet fixed relative to the external coil 108. The cochlear implant 112 includes an implantable coil 114, an implant body 134, and an elongate stimulating assembly 116 configured to be implanted in the user's cochlea. In one example, the sound processing unit 106 is an off-the-ear (OTE) sound processing unit, sometimes referred to herein as an OTE component, which is configured to send data and power to the implantable component 112. In general, an OTE sound processing unit is a component having a generally cylindrically shaped housing 111 and which is configured to be magnetically coupled to the user's head 154 (e.g., includes an integrated external magnet 150 configured to be magnetically coupled to an internal/implantable magnet 152 in the implantable component 112). The OTE sound processing unit 106 also includes an integrated external (headpiece) coil 108 (the external coil 108) that is configured to be inductively coupled to the implantable coil 114.

It is to be appreciated that the OTE sound processing unit 106 is merely illustrative of the external devices that could operate with implantable component 112. For example, in alternative examples, the external component 104 can comprise a behind-the-ear (BTE) sound processing unit configured to be attached to, and worn adjacent to, the recipient's ear. In general, a BTE sound processing unit comprises a housing that is shaped to be worn on the outer ear of the user and is connected to the separate external coil assembly via a cable, where the external coil assembly is configured to be magnetically and inductively coupled to the implantable coil 114. It is also to be appreciated that alternative external components could be located in the user's ear canal, worn on the body, etc.

Although the cochlear implant system 102 includes the sound processing unit 106 and the cochlear implant 112, as described below, the cochlear implant 112 can operate independently from the sound processing unit 106, for at least a period, to stimulate the user. For example, the cochlear implant 112 can operate in a first general mode, sometimes referred to as an “external hearing mode,” in which the sound processing unit 106 captures sound signals which are then used as the basis for delivering stimulation signals to the user. The cochlear implant 112 can also operate in a second general mode, sometimes referred as an “invisible hearing” mode, in which the sound processing unit 106 is unable to provide sound signals to the cochlear implant 112 (e.g., the sound processing unit 106 is not present, the sound processing unit 106 is powered-off, the sound processing unit 106 is malfunctioning, etc.). As such, in the invisible hearing mode, the cochlear implant 112 captures sound signals itself via implantable sound sensors and then uses those sound signals as the basis for delivering stimulation signals to the user. Further details regarding operation of the cochlear implant 112 in the external hearing mode are provided below, followed by details regarding operation of the cochlear implant 112 in the invisible hearing mode. It is to be appreciated that reference to the external hearing mode and the invisible hearing mode is merely illustrative and that the cochlear implant 112 could also operate in alternative modes.

In FIGS. 1A and 1C, the cochlear implant system 102 is shown with an external device 110, configured to implement aspects of the techniques presented. The external device 110, which is shown in greater detail in FIG. 1E, is a computing device, such as a personal computer (e.g., laptop, desktop, tablet), a mobile phone (e.g., smartphone), remote control unit, etc. The external device 110 and the cochlear implant system 102 (e.g., sound processing unit 106 or the cochlear implant 112) wirelessly communicate via a bi-directional communication link 126. The bi-directional communication link 126 can comprise, for example, a short-range communication, such as Bluetooth link, Bluetooth Low Energy (BLE) link, a proprietary link, etc.

Returning to the example of FIGS. 1A-1D, the sound processing unit 106 of the external component 104 also comprises one or more input devices configured to capture and/or receive input signals (e.g., sound or data signals) at the sound processing unit 106. The one or more input devices include, for example, one or more sound input devices 118 (e.g., one or more external microphones, audio input ports, telecoils, etc.), one or more auxiliary input devices 128 (e.g., audio ports, such as a Direct Audio Input (DAI), data ports, such as a Universal Serial Bus (USB) port, cable port, etc.), and a short-range wireless transmitter/receiver (wireless transceiver) 120 (e.g., for communication with the external device 110), each located in, on or near the sound processing unit 106. However, it is to be appreciated that one or more input devices can include additional types of input devices and/or less input devices (e.g., the short-range wireless transceiver 120 and/or one or more auxiliary input devices 128 could be omitted).

The sound processing unit 106 also comprises the external coil 108, a charging coil 130, a closely-coupled radio frequency transmitter/receiver (RF transceiver) 122, at least one rechargeable battery 132, and an external sound processing module 124. The external sound processing module 124 can be configured to perform a number of operations which are represented in FIG. 1D by an environmental classifier 131, a sound processor 133, and an own voice detector 135. Each of the environmental classifier 131, the sound processor 133, and the own voice detector 135 can be formed by one or more processors (e.g., one or more Digital Signal Processors (DSPs), one or more uC cores, etc.), firmware, software, etc. arranged to perform operations described herein. That is, the environmental classifier 131, the sound processor 133, and the own voice detector 135 can each be implemented as firmware elements, partially or fully implemented with digital logic gates in one or more application-specific integrated circuits (ASICs), partially or fully in software, etc. Although FIG. 1D illustrates the environmental classifier 131, the sound processor 133, and the own voice detector 135 as being implemented/performed at the external sound processing module 124, it is to be appreciated that these elements (e.g., functional operations) could also or alternatively be implemented/performed as part of the implantable sound processing module 158, as part of the external device 110, etc.

The environmental classifier 131 (e.g., one or more processing elements implementing firmware, software, etc.) is configured to determine an environmental classification of the sound environment (i.e., determines the “class” or “category” of the sound environment) associated with the input audio signals received at the cochlear implant 102. The environmental classifier 131 includes a decision tree, sometimes referred to as an environmental classifier decision tree that, in certain embodiments, can be trained/updated. The own voice detector 135 (e.g., one or more processing elements implementing firmware, software, etc.) is configured to perform an own voice detection (OVD) process associated with the input audio signals received at the cochlear implant 102. The own voice detector 135 includes a decision tree, sometimes referred to herein as an own voice detection decision tree, which can be trained/updated. To provide the ability to train/update the own voice detection decision tree and/or the environmental classifier decision tree, the decision trees are stored in volatile memory and exposed to, for example, other process for updating thereof. As such, the environmental classifier 131 and the own voice detector 135 are at least partially implemented in volatile memory. The environmental classification decision tree and the own voice detection decision tree can be dynamically updated on/by the device itself (e.g., cochlear implant 102), or updated using an external computing device (e.g., external device 110).

As used herein, own voice detection (OVD) generally refers to a process in which speech signals received at a hearing device, such as a cochlear implant 102, are classified as either including the “voice” or “speech” of the user (e.g., recipient) of the hearing device (referred to herein as the recipient's own voice or simply “own voice”) or a voice or speech generated by one or more persons other than the recipient (referred to herein as “external voice”).

Returning to the example of FIGS. 1A-1D, the implantable component 112 comprises an implant body (main module) 134, a lead region 136, and the intra-cochlear stimulating assembly 116, all configured to be implanted under the skin (tissue) 115 of the user. The implant body 134 generally comprises a hermetically-sealed housing 138 in which RF interface circuitry 140 and a stimulator unit 142 are disposed. The implant body 134 also includes the internal/implantable coil 114 that is generally external to the housing 138, but which is connected to the RF interface circuitry 140 via a hermetic feedthrough (not shown in FIG. 1D).

As noted, stimulating assembly 116 is configured to be at least partially implanted in the user's cochlea. Stimulating assembly 116 includes a plurality of longitudinally spaced intra-cochlear electrical stimulating contacts (electrodes) 144 that collectively form a contact array (electrode array) 146 for delivery of electrical stimulation (current) to the recipient's cochlea. Stimulating assembly 116 extends through an opening in the recipient's cochlea (e.g., cochleostomy, the round window, etc.) and has a proximal end connected to stimulator unit 142 via lead region 136 and a hermetic feedthrough (not shown in FIG. 1D). Lead region 136 includes a plurality of conductors (wires) that electrically couple the electrodes 144 to the stimulator unit 142. The implantable component 112 also includes an electrode outside of the cochlea, sometimes referred to as the extra-cochlear electrode (ECE) 139.

As noted, the cochlear implant system 102 includes the external coil 108 and the implantable coil 114. The external magnet 150 is fixed relative to the external coil 108 and the internal/implantable magnet 152 is fixed relative to the implantable coil 114. The external magnet 150 and the internal/implantable magnet 152 fixed relative to the external coil 108 and the internal/implantable coil 114, respectively, facilitate the operational alignment of the external coil 108 with the implantable coil 114. This operational alignment of the coils enables the external component 104 to transmit data and power to the implantable component 112 via a closely-coupled wireless link 148 formed between the external coil 108 with the implantable coil 114. In certain examples, the closely-coupled wireless link 148 is a radio frequency (RF) link. However, various other types of energy transfer, such as infrared (IR), electromagnetic, capacitive and inductive transfer, can be used to transfer the power and/or data from an external component to an implantable component and, as such, FIG. 1D illustrates only one example arrangement.

As noted above, sound processing unit 106 includes the external sound processing module 124. The external sound processing module 124 is configured to process the received input audio signals (received at one or more of the input devices, such as sound input devices 118 and/or auxiliary input devices 128), and convert the received input audio signals into output control signals for use in stimulating a first ear of a recipient or user (i.e., the external sound processing module 124 is configured to perform sound processing on input signals received at the sound processing unit 106). Stated differently, the one or more processors (e.g., processing element(s) implementing firmware, software, etc.) in the external sound processing module 124 are configured to execute sound processing logic in memory to convert the received input audio signals into output control signals (stimulation signals) that represent electrical stimulation for delivery to the recipient.

As noted, FIG. 1D illustrates an embodiment in which the external sound processing module 124 in the sound processing unit 106 generates the output control signals. In an alternative embodiment, the sound processing unit 106 can send less processed information (e.g., audio data) to the implantable component 112 and the sound processing operations (e.g., conversion of input sounds to output control signals 156) can be performed by a processor within the implantable component 112.

In FIG. 1D, according to an example embodiment, output control signals (stimulation signals) are provided to the RF transceiver 122, which transcutaneously transfers the output control signals (e.g., in an encoded manner) to the implantable component 112 via external coil 108 and implantable coil 114. That is, the output control signals (stimulation signals) are received at the RF interface circuitry 140 via implantable coil 114 and provided to the stimulator unit 142. The stimulator unit 142 is configured to utilize the output control signals to generate electrical stimulation signals (e.g., current signals) for delivery to the user's cochlea via one or more of the stimulating contacts (electrodes) 144. In this way, cochlear implant system 102 electrically stimulates the user's auditory nerve cells, bypassing absent or defective hair cells that normally transduce acoustic vibrations into neural activity, in a manner that causes the recipient to perceive one or more components of the input audio signals (the received sound signals).

As detailed above, in the external hearing mode the cochlear implant 112 receives processed sound signals from the sound processing unit 106. However, in the invisible hearing mode, the cochlear implant 112 is configured to capture and process sound signals for use in electrically stimulating the user's auditory nerve cells. In particular, as shown in FIG. 1D, an example embodiment of the cochlear implant 112 can include a plurality of implantable sound sensors 165(1), 165(2) that collectively form a sensor array 160, and an implantable sound processing module 158. Similar to the external sound processing module 124, the implantable sound processing module 158 can comprise, for example, one or more processors and a memory device (memory) that includes sound processing logic. The memory device can comprise any one or more of: Non-Volatile Memory (NVM), Ferroelectric Random Access Memory (FRAM), read only memory (ROM), random access memory (RAM), magnetic disk storage media devices, optical storage media devices, flash memory devices, electrical, optical, or other physical/tangible memory storage devices. The one or more processors are, for example, microprocessors or microcontrollers that execute instructions for the sound processing logic stored in memory device.

In the invisible hearing mode, the implantable sound sensors 165(1), 165(2) of the sensor array 160 are configured to detect/capture input sound signals 166 (e.g., acoustic sound signals, vibrations, etc.), which are provided to the implantable sound processing module 158. The implantable sound processing module 158 is configured to convert received input sound signals 166 (received at one or more of the implantable sound sensors 165(1), 165(2)) into output control signals 156 for use in stimulating the first ear of a recipient or user (i.e., the implantable sound processing module 158 is configured to perform sound processing operations). Stated differently, the one or more processors (e.g., processing element(s) implementing firmware, software, etc.) in implantable sound processing module 158 are configured to execute sound processing logic in memory to convert the received input sound signals 166 into output control signals 156 that are provided to the stimulator unit 142. The stimulator unit 142 is configured to utilize the output control signals 156 to generate electrical stimulation signals (e.g., current signals) for delivery to the user's cochlea, thereby bypassing the absent or defective hair cells that normally transduce acoustic vibrations into neural activity.

It is to be appreciated that the above description of the so-called external hearing mode and the so-called invisible hearing mode are merely illustrative and that the cochlear implant system 102 could operate differently in different embodiments. For example, in one alternative implementation of the external hearing mode, the cochlear implant 112 could use signals captured by the sound input devices 118 and the implantable sound sensors 165(1), 165(2) of sensor array 160 in generating stimulation signals for delivery to the user.

According to the techniques of the present disclosure, external sound processing module 124 can also include an inertial measurement unit (IMU) 170. The inertial measurement unit 170 is configured to measure the inertia of the user's head, that is, motion of the user's head. As such, inertial measurement unit 170 comprises one or more sensors 175 each configured to sense one or more of rectilinear or rotatory motion in the same or different axes. Examples of sensors 175 that can be used as part of inertial measurement unit 170 include accelerometers, gyroscopes, inclinometers, compasses, and the like. Such sensors can be implemented in, for example, micro electromechanical systems (MEMS) or with other technology suitable for the particular application.

The inertial measurement unit 170 can be disposed in the external sound processing module 124, which forms part of external component 104, which is in turn configured to be directly or indirectly attached to the body of a user. The attachment of the inertial measurement unit 170 to the user has sufficient firmness, rigidity, consistency, durability, etc. to ensure that the accuracy of output from the inertial measurement unit 170 is sufficient for use in the systems and methods described herein. For instance, the looseness of the attachment should not lead to a significant number of instances in which head movement that is consistent with a change in posture (as described below) is not identified as such nor a significant number of instances in which head movement that is inconsistent with a change in posture is not identified as such. In the absence of such an attachment, the inertial measurement unit 170 must accurately reflect the user's head movement using other techniques. The data collected by the sensors 175 is sometimes referred to herein as head motion data or motion data. As described further below, the head motion data can be utilized to differentiate, detect or predict speech-language developmental milestones of a user of a hearing device.

As also illustrated in FIG. 1D, in certain examples, a second inertial measurement unit (IMU) 180 including one or more sensors 185 is incorporated into implantable sound processing module 158 of implant body 134. Second inertial measurement unit 180 can serve as an additional or alternative inertial measurement unit to inertial measurement unit 170 of external sound processing module 124. Like sensors 175, sensors 185 can each be configured to sense one or more of rectilinear or rotatory motion in the same or different axes. Examples of sensors 185 that can be used as part of inertial measurement unit 180 include accelerometers, gyroscopes, inclinometers, compasses, and the like. Such sensors can be implemented in, for example, micro electromechanical systems (MEMS) or with other technology suitable for the particular application. For hearing devices that include an implantable sound processing module, such as implantable sound processing module 158, that includes an IMU, such as IMU 180, the techniques presented herein can be implemented without an external processor. Accordingly, a hearing device that includes an implant body 134 and lacks an external component 104 can be configured to implement the techniques presented herein.

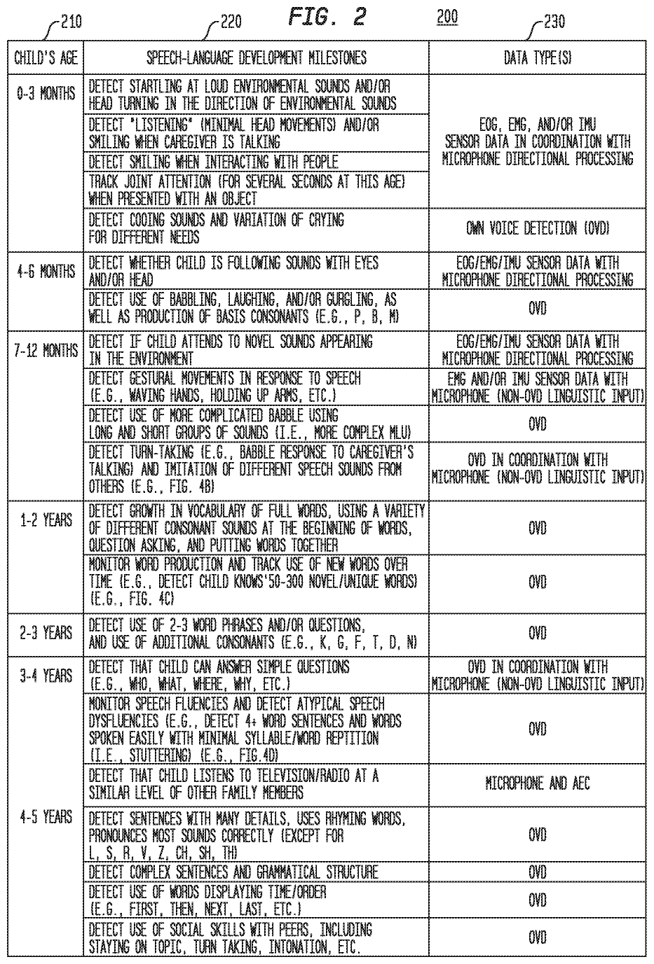

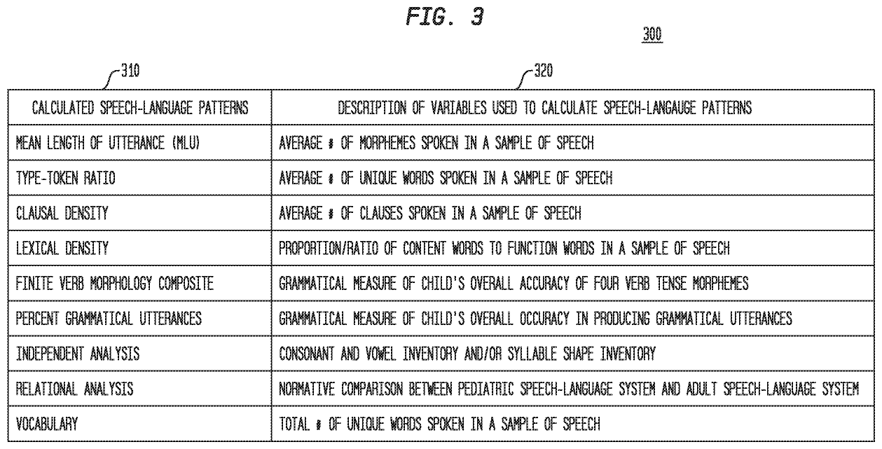

FIG. 1E is a block diagram illustrating one example arrangement for an external computing device 110 configured to perform one or more operations in accordance with certain embodiments presented herein. As shown in FIG. 1E, in its most basic configuration, the external computing device 110 includes at least one processing unit 183 and a memory 184. The processing unit 183 includes one or more hardware or software processors (e.g., Central Processing Units) that can obtain and execute instructions. The processing unit 183 can communicate with and control the performance of other components of the external computing device 110. The memory 184 is one or more software or hardware-based computer-readable storage media operable to store information accessible by the processing unit 183. The memory 184 can store, among other things, instructions executable by the processing unit 183 to implement applications or cause performance of operations described herein, as well as other data. The memory 184 can be volatile memory (e.g., RAM), non-volatile memory (e.g., ROM), or combinations thereof. The memory 184 can include transitory memory or non-transitory memory. The memory 184 can also include one or more removable or non-removable storage devices. In examples, the memory 184 can include random access memory (RAM), read only memory (ROM), EEPROM (Electronically-Erasable Programmable Read-Only Memory), flash memory, optical disc storage, magnetic storage, solid state storage, or any other memory media usable to store information for later access. By way of example, and not limitation, the memory 184 can include wired media such as a wired network or direct-wired connection, and wireless media such as acoustic, RF, infrared and other wireless media or combinations thereof. In certain embodiments, the memory 184 comprises speech-language milestone monitoring logic 195 that, when executed, enables the processing unit 183 to perform aspects of the techniques presented. In certain embodiments, the memory 184 further comprises speech-language data 196, which can include various data (e.g., table 200 of FIG. 2 and/or table 300 of FIG. 3, as further described below) that is utilized by and/or updated by the speech-language milestone monitoring logic 195.

In the illustrated example of FIG. 1E, the external computing device 110 further includes a network adapter 186, one or more input devices 187, and one or more output devices 188. The external computing device 110 can include other components, such as a system bus, component interfaces, a graphics system, a power source (e.g., a battery), among other components. The network adapter 186 is a component of the external computing device 110 that provides network access (e.g., access to at least one network 189). The network adapter 186 can provide wired or wireless network access and can support one or more of a variety of communication technologies and protocols, such as ETHERNET, cellular, BLUETOOTH, near-field communication, and RF (Radiofrequency), among others. The network adapter 186 can include one or more antennas and associated components configured for wireless communication according to one or more wireless communication technologies and protocols. The one or more input devices 187 are devices over which the external computing device 110 receives input from a user. The one or more input devices 187 can include physically-actuatable user-interface elements (e.g., buttons, switches, or dials), a keypad, keyboard, mouse, touchscreen, and voice input devices, among other input devices that can accept user input. The one or more output devices 188 are devices by which the computing device 110 is able to provide output to a user. The output devices 188 can include a display 190 (e.g., a liquid crystal display (LCD)) and one or more speakers 191, among other output devices for presentation of visual or audible information to the recipient, a clinician, an audiologist, or other user.

It is to be appreciated that the arrangement for the external computing device 110 shown in FIG. 1E is merely illustrative and that aspects of the techniques presented herein can be implemented at a number of different types of systems/devices including any combination of hardware, software, and/or firmware configured to perform the functions described herein. For example, the external computing device 110 can be a personal computer (e.g., a desktop or laptop computer), a hand-held device (e.g., a tablet computer), a mobile device (e.g., a smartphone), a surgical system, and/or any other electronic device having the capabilities to perform the associated operations described elsewhere herein.

### Own Voice Detection (OVD) Process

Before describing various example embodiments for monitoring (e.g., detecting and/or tracking) pediatric speech-language development milestones for child users (with reference to FIGS. 2, 3, 4A-4D, and 5A-5C), various techniques for performing an own voice detection (OVD) process will be described with reference to FIG. 1D.

As noted above, own voice detection (OVD) generally refers to a process in which speech signals received at a hearing device, such as a cochlear implant 102, are classified as either including the “voice” or “speech” of the recipient of the hearing device (referred to herein as the recipient's own voice or simply “own voice”), or including a voice or speech generated by one or more persons other than the recipient (referred to herein as “external voice”). A classification of speech signals received at a hearing device as either “own voice” or “external voice” using an OVD process as described herein can be helpful in, for example, providing information about how well the recipient performs with the hearing device (i.e., by indicating how much the recipient speaks and, accordingly, providing information of how “actively” the recipient uses the prosthesis). If a recipient speaks a large percentage of time, then the recipient is active and, accordingly, the recipient can understand other the speech of others (i.e., the recipient is hearing well) and the hearing device is operating as intended to improve the recipient's life. Own voice detection can enable the determination of a percentage of time a person's own voice is detected, a percentage of time an external voice is detected, and a percentage of time otherwise (e.g., in quiet or noise).

Referring again to FIG. 1D, the general operation of the external sound processing module 124 of sound processing unit 106 of cochlear implant 102, including the environmental classifier 131 and the own voice detector 135, will now be described in further detail. According to example embodiments, electrical input signals, which represent the input audio signals, are provided to the environmental classifier 131 of the external sound processing module 124 from one or more input devices (e.g., the one or more sound input devices 118 and/or the one or more auxiliary input device(s) 128) of the sound processing unit 106. The environmental classifier 131 is configured to evaluate/analyze attributes of the input audio signals (represented by the electrical input signals) and, based on the analysis, determine a “class” or “category” of the sound environment associated with the input audio signals. The environmental classifier 131 can be configured to categorize the sound environment into a number of predetermined sound environment classes/categories. In one illustrative example, the environmental classifier 131 is configured to classify or categorize the sound environment into one of five (5) classes or categories, including “Speech,” “Speech in Noise,” “Quiet,” “Noise,” and “Music,” although other categories are possible.

The environmental classifier 131 operates to determine a sound environment class or category for the set of input audio signals by calculating, in real-time, a plurality of time-varying features from the input audio signals and analyzing the calculated time-varying features using a type of decision tree. As a result of the analysis, the environmental classifier 131 determines the most likely sound environment class or category (i.e., either “speech,” “speech in noise,” “quiet,” “noise,” or “music”) for the set of input audio signals. The environmental classifier 131 includes a number of processes/algorithms that calculate time-varying features from the input audio signals. The environmental classifier 131 also includes an environmental classification decision tree, which uses all or some of these time-varying features as inputs to classify or categorize the input audio signals into one of the predetermined sound environment classes or categories. The decision tree includes a number of hierarchical or linked branches/nodes that each perform evaluations, comparisons, or checks using at least one of the time-varying features to determine the sound environment classification at the branch ends (leaves). That is, the decision tree traverses its “branches” until it arrives at a “leaf” and decides “speech,” “speech in noise,” “quiet,” “noise,” or “music.” As noted, own voice detection is only relevant for the predetermined sound environment classes or categories of the input audio signals, as determined by the environmental classifier 131, that include speech, namely the “Speech” and “Speech in Noise” categories (sometimes collectively referred to herein as speech classes or categories). As such, when the environmental classifier 131 determines the input audio signals are associated with a speech class (e.g., either “speech” or “speech in noise”), then the input audio signals are provided to the own voice detector 135, and further classified by the own voice detector 135 as either being “own voice” (i.e., the hearing device recipient is speaking within the set of input audio signals) or as “external voice” (i.e., someone other than the hearing device recipient is speaking within the set of input audio signals.

When the own voice detector 135 receives the input audio signals that have been classified/categorized by the environmental classifier 131 as being associated with a speech class/category (e.g., “speech” or “speech in noise”), the own voice detector 135 operates by calculating, in real-time, a plurality of time-varying features from the input audio signals (as represented by the electrical input signals) and analyzing the calculated time-varying features using a type of decision tree. The own voice detector 135 can calculate a number of different time-varying features (e.g., such as volume level, proximity level, amplitude modulations, modulation depth, spectral profile, harmonicity, amplitude onsets, etc.) from the input audio signals, and the specific features can vary for different implementations. As a result of the analysis, the own voice detector 135 determines the most likely voice/speech category (i.e., either “own voice” or “external voice”) for the set of input audio signals. The own voice detector 135 includes a number of processes/algorithms that calculate time-varying features from the input audio signals. The own voice detector 135 also includes an own voice detection decision tree, which uses all or some of these time-varying features as inputs to classify the input audio signals as either “own voice” or “external voice.” The OVD decision tree includes a number of hierarchical or linked branches/nodes that each perform evaluations, comparisons, and/or checks using at least one of the time-varying features to determine the voice/speech classification (i.e., own voice or external voice) at the branch ends (leaves). That is, the decision tree traverses its “branches” until it arrives at a “leaf” and decides “own voice” or “external voice.”

As noted, the environmental classifier 131 and the own voice detector 135 each make use of decision trees. For ease of illustration and description, the environmental classifier 131 and the own voice detector 135, as well as the corresponding decision trees, are described as separate functional entities. However, it is to be appreciated that the environmental classifier 131 and the own voice detector 135 can be implemented as a single element using two decision trees or decision tree segments that operate in a parent/child relationship to generate the different classifications (i.e., the environmental classification and the own voice classification).

### Monitoring Speech-Language Development Milestones for Pediatric Users

Next, various techniques for detecting and speech-language development milestones for pediatric (child) users will be described with reference to FIGS. 2, 3, 4A-4D, and 5A-5C.

Children generally follow certain milestones as they develop their speech-language abilities over time. Professionals, such as speech-language pathologists, commonly record speech manually and calculate language patterns to assess language complexity and assess whether children are meeting developmental milestones. However, this process is often time consuming and cumbersome, and is not available to all children. As such, it is common for speech-language delays to go undetected until a child enters an educational setting where professionals may or may not be trained to identify the warning signs. At this point, a child with speech-language delays may already be at risk of being negatively impacted socially and/or educationally. Speech-language delays are then identified by formal professional assessment. These formal assessments can be stressful and time-consuming, as well as have associated costs to the user's family and healthcare system.

Children with hearing impairment are at elevated risk for developing speech-language delays, even after receiving hearing intervention (e.g., cochlear implantation). Speech-language delays often go undetected until children become socially or educationally impacted. Early detection and intervention are critical for resolving speech-language delays before children are negatively impacted.

As noted above, user devices, such as cochlear implant system 102 of FIGS. 1A-1D, hearing devices, smartwatches, etc., can include microphones, inertial measurement units (IMUs) or other sensors, which can obtain/capture data, such as acoustic data and/or sensor data (motion data), that can be utilized to detect and track one or more aspects of pediatric speech-language development. For example, an IMU can provide sensor data indicating movement data of a user. In some situations, however, sensor data (motion data) alone can be insufficient to differentiate between different activities or milestones and/or to determine whether a user has reached a particular speech-language developmental milestone. Thus, the microphone(s) or other acoustic detectors can capture and provide acoustic data that can be used to contextualize sensor data (motion data) acquired by the IMU(s) (e.g., using microphone directional processing) in order to differentiate, determine or predict various speech-language developmental milestones.

According to an aspect of the present disclosure, a systems, methods, etc. are provided that automate a process for calculating speech-language patterns to assess language complexity and determine whether children are meeting developmental milestones. The techniques presented herein can, in certain examples, monitor a user's speech-language development based on Own Voice Detection (OVD) data (as described above) or other acoustic data. The techniques presented herein can, in certain examples, monitor a user's speech-language development based on other sensor data (e.g., IMU data), potentially in combination with the acoustic data.

In some example embodiments, a user's speech-language development can be characterized/determined, and then compared to age-specific normative values to track critical speech-language milestones. Based on age and speech-language abilities, the system can identify a user as meeting developmental milestones or as being delayed and/or at-risk of developmental delay. This information can be provided to caretakers and/or delivered to professionals. If the user is delayed or at risk of developmental delay, then the system can provide recommendations, such as seeking early intervention.

If, for example, a child's determined speech-language development is on-track for the child's age, the caretaker(s) can happily track their child's progress while validating their own perceptions. If development is delayed, caretaker(s) can seek early intervention and track progress. This feature would also improve accessibility of care to rural individuals and others that lack easy access to care.

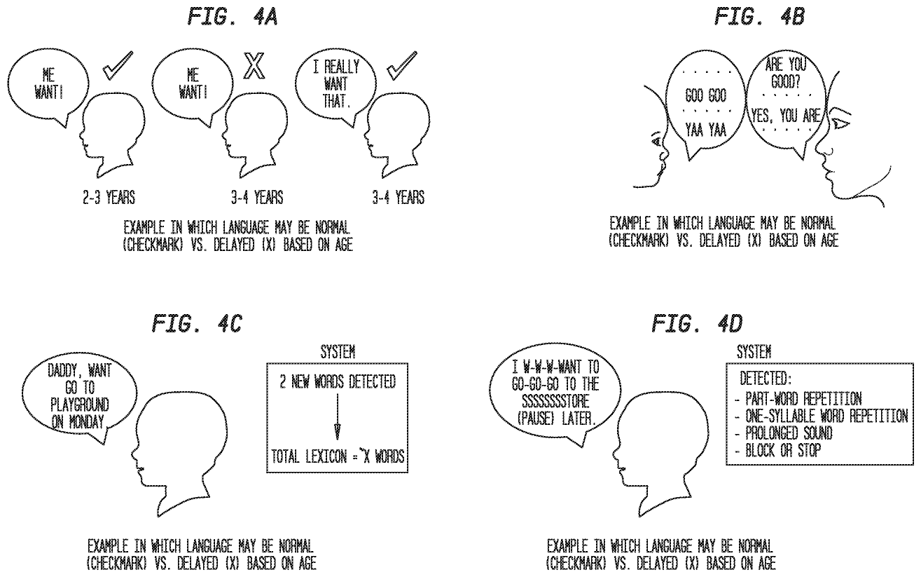

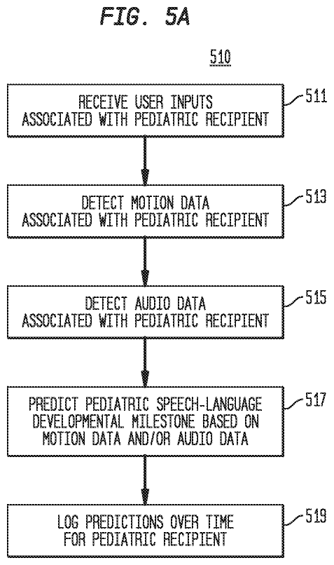

In some example embodiments, described below with reference to FIG. 2, OVD acoustic input can be combined with other microphone data (e.g., non-OVD linguistic input) and/or sensor input to detect and track speech-language developmental milestones for different age ranges of children. Examples of other sensor input can include, but are not limited to, electrooculography (EOG), electromyography (EMG), inertial measurement unit (IMU), and/or combinations of such sensor data. In some example embodiments, described below with reference to FIG. 3, various common calculated speech-language patterns can be used by the system to assess speech-language development. In an example embodiment shown in FIG. 4A, language complexity of a given child can be compared to age-specific normative values (for that child's age or another age range) to determine if the child's speech-language development is normal/on-track (checkmark) or delayed (X) based on age-specific normative values. A plurality of pediatric speech-development milestones and accompanying acoustic data (e.g., OVD information) are described below with reference to FIGS. 4B, 4C, and 4D, respectively.

Referring first to FIG. 2, shown is a table illustrating various example speech-language development milestones, and accompanying sensor data and/or acoustic data, for different age ranges of children. This data could, for example, be used by the system of FIGS. 1A-1D (or other devices/systems) to assess pediatric speech-language development. Illustrated in FIG. 2 is an exemplary table 200 with a column 210 indicating an expected month (or age range) at which children are expected to reach the pediatric speech-language developmental milestones, a column 220 indicating a description of various pediatric speech-language developmental milestones, and a column 230 indicating various data type(s), such as EOG, EMG, and/or IMU sensor data (motion data) that can be detected when a pediatric user is performing an action associated with a pediatric speech-language developmental milestone, and/or acoustic data (microphone data, OVD data, non-OVD input) that can be associated with the action associated with the pediatric speech-language developmental milestone.

Generally, there are four example sets of data type(s) in table 200 of FIG. 2: (1) sensor data (e.g., motion data) combined with microphone directional processing, (2) OVD data only, (3) OVD data combined with other microphone data (e.g., non-OVD linguistic input), and (4) sensor data (e.g., motion data) combined with other microphone data (e.g., non-OVD linguistic input), although example embodiments are not limited thereto. In some example embodiments, different example data types can be more or less useful for certain ages or age ranges (e.g., sensor data is more useful from 0 months to 1 year, whereas OVD data becomes more useful as the child ages from 1 year to 5 years), as further described below.

Birth to 3 months of age. As illustrated in table 200 shown in FIG. 2, the system can use EOG, EMG, and/or IMU sensor data in coordination with microphone directional processing to detect startling at loud environmental sounds and/or head turning in the direction of environmental sounds. The system can use EOG, EMG, and/or IMU data in coordination with microphone directional processing to detect ‘listening’ (minimal head movements) or smiling when a caregiver is talking. The system can use OVD to detect cooing sounds and variation of crying for different needs. The system can use EOG, EMG, and/or IMU data in coordination with microphone directional processing to detect smiling when interacting with people. The system can use EOG, EMG, and/or IMU data in coordination with microphone directional processing to track joint attention (for several seconds at this age) when presented with an auditory object.

4 to 6 months of age. As illustrated in table 200 shown in FIG. 2, the system can use EOG, EMG, and/or IMU data in coordination with microphone directional processing to detect whether user is following sounds with their eyes and/or head. The system can use OVD to detect use of babbling, laughing, and gurgling, as well as production of /p/, /b/, and /m/ consonants.

7 to 12 months of age. As illustrated in table 200 shown in FIG. 2, the system can use EOG, EMG, and/or IMU data in coordination with microphone directional processing to detect if user attends to novel sounds appearing in the environment. EMG and IMU in coordination with microphone (non-OVD linguistic input) can detect gestural movements, such as waving or holding up arms, in response to speech. The system can use OVD to detect use of more complicated babble using long and short groups of sounds (i.e., more complex MLU). The system can use OVD and microphone (non-OVD linguistic input) to detect turn-taking (e.g., babble response to caregiver's talking) and imitation of different speech sounds from others. FIG. 4B depicts an example of how the system of FIGS. 1A-1E uses acoustic data (OVD information compared to non-OVD lexical input) to detect and track normal turn-taking and speech sound imitation, as developmentally appropriate for this age range (~7-12 months old).

1 to 2 years of age. As illustrated in table 200 shown in FIG. 2, the system can use OVD to detect growth in vocabulary of full words, using a variety of different consonant sounds at the beginning of words, question asking, and putting words together. The system can use OVD to detect the child knows ~50-300 novel words. FIG. 4C depicts an example of how the system of FIGS. 1A-1E uses acoustic data (OVD information) to detect and monitor novel word production, and to track use of new words and vocabulary grown over time, as developmentally appropriate for this age range (~1-2 years old).

2 to 3 years of age. As illustrated in table 200 shown in FIG. 2, the system can use OVD to detect use of 2-3 word phrases/questions and use of /k/, /g/, /f/, /t/, /d/, and /n/ consonants.

3 to 4 years of age. As illustrated in table 200 shown in FIG. 2, the system can use OVD and microphone (non-OVD linguistic input) to detect that they answer simple questions (i.e., Who? What? Where? And Why? Questions). Microphone and acoustic environment classification (AEC) to detect that user is listening to television/radio at a similar level of other family members. The system can use OVD to detect 4+ word sentences and words spoken easily with minimal syllable/word repetition (i.e., stuttering). FIG. 4D depicts an example of how the system of FIGS. 1A-1E uses acoustic data (OVD information) to monitor speech fluency and detect dysfluencies atypical of user's age, as developmentally appropriate for this age range (~3-4 years old).

4 to 5 years of age. As illustrated in table 200 shown in FIG. 2, the system can use OVD to detect sentences with many details, pronounces most sounds correctly (except for 1, s, r, v, z, ch, sh, and th), uses rhyming words. The system can use OVD to detect complex sentences and grammatical structure. The system can use OVD to detect use of words displaying time and order (first, then, next, last, etc.). The system can use OVD to detect use of social skills with peers, including staying on topic, turn taking, intonation, etc.

In some example embodiments, an estimated pediatric speech-language developmental milestone associated with the child user can be logged over time to determine an age at which the child user is reaching the pediatric speech-language developmental milestones or to predict when a child user can reach future developmental milestones. For example, based on the sensor data (motion data) and the acoustic data (microphone data) associated with a child user, the system can predict that the child user has reached a particular speech-language developmental milestone, and an indication that the child user has reached the particular speech-language developmental milestone can be logged in a log along with an age (e.g., in days, months, and/or years) of the child user. An age when the pediatric user reaches speech-language developmental milestones can be compared to an average age (or age range) when children reach the developmental milestones to determine whether the pediatric user is on target for reaching the milestones or is approaching the milestones at a delayed rate.

The pediatric speech-language developmental milestones, ages or age ranges (e.g., months, years), sensor data (e.g., motion data), and acoustic data (e.g., microphone data, such as OVD information and/or non-OVD lexical input) described above with respect to table 200 of FIG. 2 are exemplary and non-limiting. Additional speech-language developmental milestones associated with a pediatric user can be experienced and logged. In addition, different sensor data and/or acoustic data can be associated with the pediatric speech-language developmental milestones described above with respect to FIG. 2.

As mentioned above, when using OVD acoustic data, the system can also calculate various speech-language patterns to assess speech-language development of the child users. FIG. 3 is a table illustrating various examples of common calculated speech-language patterns that are used by the system of FIGS. 1A-1E to assess pediatric speech-language development, according to some example embodiments. Illustrated in FIG. 3 is an exemplary table 300 with a column 310 indicating common calculated speech-language patterns used to assess speech-language development, and a column 320 indicating a description of different variables used to calculate the speech-language patterns 310.

Referring to table 300 shown in FIG. 3, common calculated speech-language patterns used to assess speech-language development can include: (1) Mean length of utterance (MLU): the average number of morphemes spoken in a sample of speech; (2) Type-token ratio: the average number of unique words spoken in a sample of speech; (3) Clausal density: the average number of clauses spoken in a sample of speech; (4) Lexical density: the proportion of content words to function words in a sample of speech; (5) Finite verb morphology composite: grammatical measure of children's overall accuracy of four verb tense morphemes; (6) Percent grammatical utterances: grammatical measure of children's overall accuracy in producing grammatical utterances; (7) Independent analysis: consonant and vowel inventory/syllable shape inventory; (8) Relational analysis: normative comparison between a child's speech-language system and an adult's speech-language system; and (9) Vocabulary: the number of unique words known.

In an example embodiment, the system can detect phonemic usage (e.g., the child just voiced the “z” sound for the first time). In another example embodiment, speech patterns can also be assessed for atypical dysfluencies, such as stuttering, characterized by part-word repetitions (e.g., “c-c-c-cat), one-syllable word repetitions (e.g., “be-be-be”), prolonged sounds (“ffffffor”), and blocks or stops (e.g., “I want a (pause) snack”). These characteristic dysfluencies can be distinguished from normal dysfluencies (that are not indicative of stuttering), such as interjection (e.g., “I am umm hungry”), repeating whole words (e.g., “peanut-peanut butter”), repeating phrases (e.g., she is-she is tall”), revision (e.g., “I was-I am 2”), or thought abandonment (e.g., “he is . . . nevermind”).

While specific speech-language developmental milestones used clinically will potentially vary based on future research findings, the building blocks (e.g., MLU, type-token ratio, etc.) can likely still be applied to future standards. Speech-language delay/disorder is the most prevalent early child disorder, yet there is a lack of focus within the medical community for speech-language disorders as well as hearing loss in children. Children with hearing loss are more likely to have speech-language delay due to the absence of critical speech-language input as an infant and/or child. Early intervention, which is crucial for children who fall into the category of having hearing loss, relies on early detection and identification. The first 12 months of life are imperative for speech-language learning in children. By the age of 6 months, a child should be producing consonant-vowel babbles in various forms, and at 1 year of age the child should demonstrate a small vocabulary of core words relative to their daily life (e.g., mom, dad, dog, ball, etc.).

According to an example embodiment, a lack of these verbal indicators can be detected by OVD through, for example, a mobile application on a smartphone. Other early markers of a possible speech-language delay before the age of 12 months could include lack of turn taking, lack of joint attention, lack of babbling, lack of eye contact, lack of engagement through smiling, and lack of using gestures while communicating. Using EOG, EMG, and/or IMU data to capture these indicators, aside from language alone, can help with earlier identification, and therefore early intervention.

As mentioned above, information regarding speech-language development can be logged by the system. The system can flag users as meeting speech-language developmental milestones or being at-risk of being developmentally delayed. In an example embodiment, data including but not limited to developmental milestones and/or suspected delays can be viewed by the caretaker(s), such as by using the mobile application on the smartphone. If suspected delays are detected, then the system can prompt the caretaker(s) to seek early intervention. Such data can also be relayed directly to a professional (e.g., a speech-language pathologist, a teacher, an occupational therapist, etc.) using secured cloud services, for example. Datalogging acoustic data (e.g., OVD information and/or non-OVD lexical input), as well as other sensor data (e.g., EOG, EMG, and/or IMU) into the mobile application will not only allow for early detection and intervention, but will also increase accessibility of professional therapies and guidance, for all families, particularly those in rural areas.

In an example embodiment, if delays are identified, then acoustic environment classification (AEC) data could be used to provide potential mitigating factors (e.g., child is not getting enough “speech in quiet” practice, which is optimal for speech-language development) and recommend countermeasures (e.g., speak with your child one-on-one more). Providing a mobile application with OVD, where datalogging is occurring for all verbal communications throughout the day, would be immensely helpful to professionals who could use the data to calculate speech-language measures in a child's naturalistic environment, as opposed to a structured therapy environment, where a language sample can be more accurate of the child's abilities. This example embodiment could also help bridge the gap of communication between caregivers and professionals, offering more transparency about therapy and milestone expectations, as well as between professionals (e.g., audiologist and speech-language pathologist).

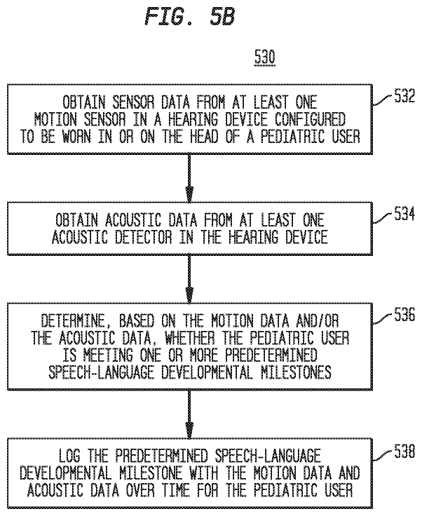

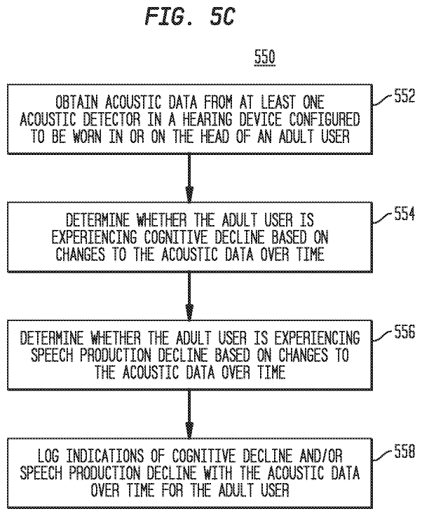

FIG. 5A is a flowchart 510 illustrating an example method for identifying pediatric speech-language development milestones based on sensor data and/or acoustic data, according to an example embodiment. The process flow of flowchart 510 begins in operation 511 where inputs associated with a pediatric user of a hearing device configured to be worn in or on the head of the pediatric user can be received. For example, a device, such as external computing device 110 of FIGS. 1A-1E can receive inputs associated with the pediatric user. The inputs can include, for example, a birthdate/age of the pediatric user, and an indication of whether the pediatric user is experiencing any developmental delay in reaching pediatric speech-language developmental milestones or is experiencing a speech-language disorder. Additional and/or different inputs associated with the pediatric user can be received. In some situations, no inputs associated with the pediatric user can be received.

In operation 513, sensor data (motion information) associated with the hearing device is detected. For example, EOG sensors, EMG sensors and/or IMU sensors included in the hearing device, such as those included in the inertial measurement unit 170 and/or the inertial measurement unit 180 of FIG. 1D, can be used to obtain sensor data (motion information) associated with the hearing device. The sensor data can track eye, mouth, head and/or body movements that are indicative of attentional focus of the pediatric user.

In operation 515, audio data (e.g., OVD information and/or microphone data (non-OVD linguistic input)) associated with the hearing device is detected. For example, acoustic detectors or microphones included in the hearing device, such as the sound input devices 118 of FIG. 1D, can be used to obtain audio data associated with the pediatric user. The audio data can include, for example, OVD information associated with a voice of the pediatric user.

In operation 517, the system can then predict a speech-language developmental milestone associated with the pediatric based on the sensor data (motion information), the audio data (e.g., OVD information and/or microphone data (non-OVD linguistic input)), or a combination thereof. For example, a device, such as external computing device 110 of FIGS. 1A-1E, can predict the speech-language developmental milestone of the pediatric user using the sensor data (motion information). Additionally or alternatively, the external device 110 can predict the speech-language developmental milestone of the pediatric user using the audio data (e.g., OVD information). A current age of the pediatric user and the indication of whether the pediatric user is experiencing a delay in reaching speech-language developmental milestones or a speech-language disorder can be used to interpret or adjust a developmental milestone of the pediatric user.

In one example, operation 517 can include comparing the sensor data (motion information) and/or the acoustic data to age-specific normative values for respective pediatric speech-language developmental milestones, and determining that the user is on-track (normal) when a result of the comparing is consistent with (matches or corresponds to) the age-specific normative values in connection with one of the pediatric speech-language developmental milestones. In another example, operation 517 can include comparing the sensor data (motion information) and/or the acoustic data to age-specific normative values for respective pediatric speech-language developmental milestones, and determining that the user is delayed or at risk of developmental delay when a result of the comparing is inconsistent with (does not match or correspond to) the age-specific normative values in connection with one of the pediatric speech-language developmental milestones. For example, the pediatric speech-language developmental milestone can be predicted using table 200 of FIG. 2. In some example embodiments, the pediatric speech-language developmental milestone can be predicted using table 300 of FIG. 3 (common calculated speech-language patterns).

In one example embodiment, a machine learning algorithm can be used to predict the pediatric speech-language developmental milestones based on sensor data (motion information) and/or acoustic data (e.g., OVD information and/or microphone data (non-OVD linguistic input)) associated with a large sample of pediatric users (e.g., a Big Data set). In this embodiment, the sensor data (motion information) and the acoustic data (e.g., OVD information or other microphone data) in table 200 of FIG. 2 can be updated based on the machine learning algorithm. Likewise, the calculated speech-language patterns in table 300 of FIG. 3 can be updated based on the machine learning algorithm.

In operation 519, the predicted speech-language developmental milestones are logged over time for the pediatric user. For example, the external device 110 (or the external computing device 190) can log an age at which the pediatric user has reached the predetermined pediatric speech-language developmental milestones. The log can be used to determine whether the pediatric user is meeting age-appropriate speech-language developmental milestones. Parents or caregivers can be informed whether the pediatric user is tracking to standard developmental milestones. If the pediatric user is not meeting the standard developmental milestones, then additional diagnoses or assistance can be provided. For example, delays or other pediatric speech-language developmental problems can be detected by the device and communicated to a clinician or a caregiver.

FIG. 5B is a flowchart 530 illustrating another method for identifying pediatric speech-language development milestones based on sensor data and/or acoustic data, according to another example embodiment. The process flow of flowchart 530 begins in operation 532 where sensor data (motion information) is detected from at least one motion sensor in a hearing device configured to be worn in or on the head of a pediatric user. For example, sensors included in a hearing device, such as those included in the inertial measurement unit 170 and/or the inertial measurement unit 180 of FIG. 1D, can be used to obtain sensor data (motion information) associated with the hearing device. The hearing device can be an external or wearable hearing device, or a combination thereof.

In operation 534, acoustic data is detected from at least one acoustic detector in the hearing device. For example, microphones or sound input devices included in the hearing device, such as those included in one or more sound input devices 118 of FIG. 1D, can be used to obtain acoustic data associated with the pediatric user of the hearing device. The acoustic data can include own voice detection (OVD) information associated with a voice signal of the pediatric user.

In operation 536, the system determines, based on the sensor data (motion information) and/or the acoustic data (OVD information and/or other microphone data (e.g., non-OVD lexical input)), whether the pediatric user is meeting one or more predetermined speech-language developmental milestones. For example, a device, such as external device 110 of FIGS. 1A-1E, can determine whether the pediatric user is meeting a speech-language developmental milestone using the sensor data (motion information), the acoustic data (e.g., OVD information), or a combination thereof. A current age of the pediatric user and the indication of whether the pediatric user is experiencing a delay in reaching speech-language developmental milestones or a speech-language disorder can be used to interpret or adjust a developmental milestone of the pediatric user.

In one example, operation 536 can include comparing the sensor data and/or the acoustic data to age-specific normative values for respective pediatric speech-language developmental milestones, and determining that the user is on-track (normal) when a result of the comparing is consistent with (matches or corresponds to) the age-specific normative values in connection with one of the pediatric speech-language developmental milestones. In another example, operation 536 can include comparing the motion data and/or the acoustic data to age-specific normative values for respective pediatric speech-language developmental milestones, and determining that the user is delayed or at risk of developmental delay when a result of the comparing is inconsistent with (does not match or correspond to) the age-specific normative values in connection with one of the pediatric speech-language developmental milestones. For example, whether the pediatric user is meeting a pediatric speech-language developmental milestone can be determined using information in table 200 of FIG. 2, using information in table 300 of FIG. 3 (common calculated speech-language patterns), or a combination thereof.

In one example embodiment, a machine learning algorithm can be used to determine whether the pediatric user is meeting the speech-language developmental milestones based on sensor data (motion information) and/or acoustic data (e.g., OVD information and/or microphone data (non-OVD linguistic input)) associated with a large sample of pediatric users (e.g., a Big Data set). In this embodiment, the sensor data (motion information) and the acoustic data (e.g., OVD information or other microphone data) in table 200 of FIG. 2, and/or the calculated speech-language patterns in table 300 of FIG. 3, can be updated based on the machine learning algorithm.

In operation 538, the system logs whether the pediatric user is meeting the predetermined speech-language developmental milestones in a log with the sensor data and/or the acoustic data over time. For example, the external computing device 110 can log that the pediatric user is normal or on-track with respect to a particular speech-language developmental milestone based on the current age of the pediatric user. Additionally or alternatively, the external device 110 can log that the pediatric user is delayed or at risk of developmental delay with respect to a particular speech language-developmental milestone based on the current age of the pediatric user.

Using the techniques of the present disclosure (e.g., flowchart 510 of FIG. 5A and/or flowchart 530 of FIG. 5B), delays or other speech-language developmental problems associated with pediatric users can be detected early and rehabilitation can be applied. Furthermore, these techniques can be used to coach family members to provide additional opportunities for rehabilitation and the techniques can be used to track whether the interventions are having a desired impact. Additionally, the log of pediatric speech-language developmental milestones and current sensor data (motion data) and acoustic data (OVD information and/or non-OVD lexical input) can be used to predict when a pediatric user is about to reach a new speech-language developmental milestone.

### Detecting Speech-Language Changes in Adult Users

On the other end of the spectrum, the system described above with reference to FIGS. 1A-1E (or other systems/devices) and some of the techniques for monitoring (e.g., tracking) speech-language developmental milestones for child users described above with reference to FIGS. 2, 3, 4A-4D, and 5A-5B can be utilized in a different manner for adult users as described below with reference to FIG. 5C. According to another example embodiment, speech-language changes in adult users can be detected by the system as potential predictors of cognitive decline or speech production decline.

In the case of “cognitive decline,” older adults often experience “tip-of-the-tongue” word-finding failures. The system can use OVD information to detect these events in older adults, similar to detecting part-word and block/stop stuttering for children as described above. Word-finding failures can be distinguished from traditional stuttering in that traditional stuttering is unlikely to develop in adults. Detecting an increase in word-finding failures over time can be indicative of cognitive decline. While there is no cure for age-related cognitive decline, early detection will allow users and/or caretakers to implement strategies to mitigate the effects of cognitive decline.

In the case of “speech production decline,” in advanced age the tissues of the speech production system (e.g., vocal folds, related musculature, etc.) experience neuromuscular changes that can cause weakening and reduced coordination. Speech-language therapy can be effective in slowing or reversing these age-related speech production declines. The system can use OVD information to detect several early detectors of speech production decline, including but not limited to (1) decreased amplitude, (2) increased phonological errors, and/or (3) slowing of speech.

“Decreased amplitude” refers to changes to (reduction of) the root-mean-squared amplitude over time that can be indicative of speech production decline related to decreases in respiratory capacity. “Increased phonological errors” refers to phonological errors that can be detected by consonant substitutions (e.g., “par” when meaning to say “bar”). Even when producing the intended phoneme, older adults with speech production decline have distinct acoustics from younger adults, such as atypical fricative (e.g., “sh”, “s”, “f”, “v”, and/or “z” sounds) spectral characteristics, due to reduced precision of the speech articulators. “Slowing of speech” refers to changes to the modulation characteristics (increase in low-frequency modulations and decrease in high-frequency modulations) over time that can be indicative of overall speech production decline.

There can be a tradeoff between (2) phonological errors and (3) slowing rate of speech in that individuals with speech production decline can slow down their speech to reduce the number of phonological errors produced. Therefore, in some example embodiments, the system can detect speech production decline through a preponderance of increased phonological errors in the absence of slowing speech, slowing of speech without an increase in phonological errors, or a combination of these two factors.

FIG. 5C is a flowchart 550 illustrating an example method for detecting speech-language changes as potential predictors of cognitive decline or speech production decline based on acoustic data (OVD information), according to yet another example embodiment. The process flow of flowchart 550 begins in operation 552 where acoustic data is obtained from at least one acoustic detector in a hearing device configured to be worn in or on the head of an adult user. For example, acoustic data can be obtained from microphones or sound input devices included in the hearing device, such as those included in one or more sound input devices 118 of FIG. 1D. The acoustic data can include own voice detection (OVD) information associated with a voice signal of the adult user. In some example embodiments, the flowchart then proceeds to operation 554. In some other example embodiments, the flowchart 550 can proceed directly to operation 556 (without performing operation 554) after operation 552.

In operation 554, the system determines, based on the acoustic data (including OVD information and/or other microphone data (e.g., non-OVD lexical input)), whether the adult user is experiencing cognitive decline based on changes to the acoustic data over time (e.g., increasing or decreasing values). In some example embodiments, the flowchart 550 then proceeds to operation 556. In some other example embodiments, the flowchart 550 can proceed directly to operation 558 (without performing operation 556) after operation 554.

In operation 556, the system determines, based on the acoustic data (including OVD information and/or other microphone data (e.g., non-OVD lexical input)), whether the adult user is experiencing speech production decline based on changes to the acoustic data over time (e.g., increasing or decreasing values).

For example, a device, such as external computing device 110 of FIGS. 1A-1E, can determine whether the adult user is experiencing cognitive decline and/or speech production decline using the acoustic data (e.g., OVD information). In operation 554 and/or operation 556, the obtained acoustic data (e.g., OVD information or other microphone data) can be compared to user information stored in a user-specific table (not shown in figures) to determine whether the adult user is experiencing cognitive decline and/or speech production decline. The user information can include initial baseline values for the adult user (e.g., captured from one or more initial executions of the method of flowchart 550) and/or historical values for the adult user (e.g., captured from one or more subsequent executions of the method of flowchart 550 prior to the current execution).

In operation 558, the system logs indications of the cognitive decline and/or the speech production decline in a log with the acoustic data over time for the adult user. For example, the external computing device 110 can log that the adult user is experiencing cognitive decline based on changes to speech-language patterns associated with the adult user over time. Additionally or alternatively, the external computing device 110 can log that the adult user is experiencing speech production decline based on changes with respect to amplitude (decreased amplitude), phonological errors (increased phonological errors), rate of speech (slowing of speech), or a combination thereof (e.g., a preponderance of phonological errors in the absence of slowing speech, or slowing speech without an increase in phonological errors).

Early detection of cognitive decline and/or speech production decline using the techniques of the present disclosure described above will allow elderly or adult users and/or caretakers to seek speech-language therapy and/or implement other strategies to mitigate the effects of speech production decline and/or cognitive decline.

### Summary and Illustrative Advantages

In summary, the present invention provides a new system and methods for monitoring and managing speech-language development for pediatric users and/or speech-language decline for adult users. The system includes technologies for analyzing aspects of the user's response, such as eye, mouth, head or body movement or other indicators of the user's attentional focus. Exemplary technologies for analyzing the user's response can include EOG, EMG, and IMU (movement sensors), among others (e.g., microphones). This new combination of user responsive factors (indicative of the user's attentional focus) with acoustic inputs (including the voice of the user) provides a more comprehensive and reliable system for analyzing speech-language development in the context of pediatric patients (as well as adult patients in another example embodiment). Advantageously, the system can also be integrated into an implantable medical device, such as a hearing prosthesis (e.g., a cochlear implant), to improve accessibility to diagnostic co-morbidities as well as improving accessibility to multi-modal (and potentially synergistic) treatment strategies.

The proposed techniques and example embodiments thereof are unique is that it utilizes acoustic data (e.g., OVD information) and non-acoustic sensor data (e.g., motion information captured via EOG, EMG, and/or IMU sensors) to more comprehensively assess speech-language abilities at all stages of the developmental trajectory. Example embodiments describe specific linguistic characteristics extracted by the system and how information can be used for determining a user's speech-language development (or decline). Some example embodiments incorporate non-acoustic input (e.g., IMU sensor data) to detect user attention to novel sound sources or to track a moving sound source. The system does not require the use of an external recording device, and can optionally include other sensors to track the activity of the children with regard to their general activity and developmental level. In an example embodiment of a cochlear implant worn in/on the head, the system can have access to other sensors (e.g., EOG, EMG, IMU) to more comprehensively assess speech-language abilities (e.g., a user attending to novel sound sources). For example, the system can track the location or movement of a sound source relative to head movement and/or eye movement of the recipient. Advantageously, the system provides the ability to objectively measure the internal or attentional focus of a child and/or responsive factors, and to make correlations with relevant acoustic inputs (e.g., direction of incoming sound, linguistic content, especially the user's own voice).

In addition, some example embodiments can involve a machine learning approach, wherein the system collects various speech-language data (e.g., acoustic data and/or motion data from other sensors) from many individuals to yield a Big Data set. The users'data can then be classified either by a professional, caretaker, or the user on their speech-language abilities (e.g., developmentally delayed, having characteristic “deaf speech,” fluency disorders, normal, etc.). Then acoustic features are extracted, and a supervised machine-learning classification model is used on other recipient's data to classify their speech-language abilities and provide rehabilitations recommendations, if appropriate. Thus, one key distinction from existing technologies that use sensor data and machine learning in general is the proposed invention's focus on various factors that are relevant to speech-language development, specifically.

### Example Use Cases and Applications

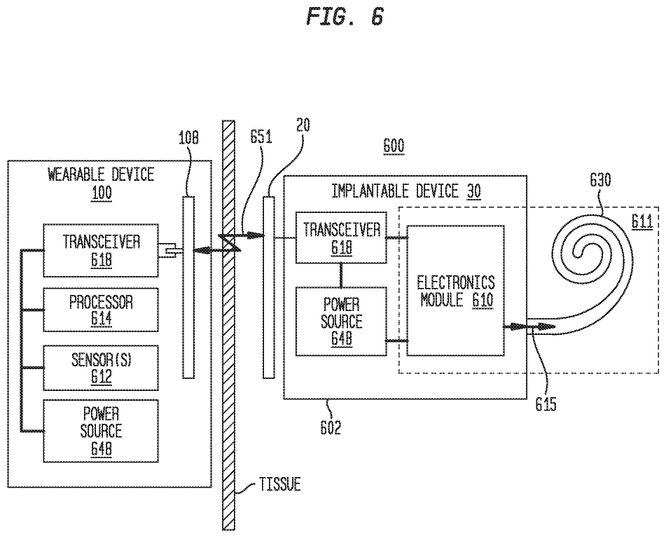

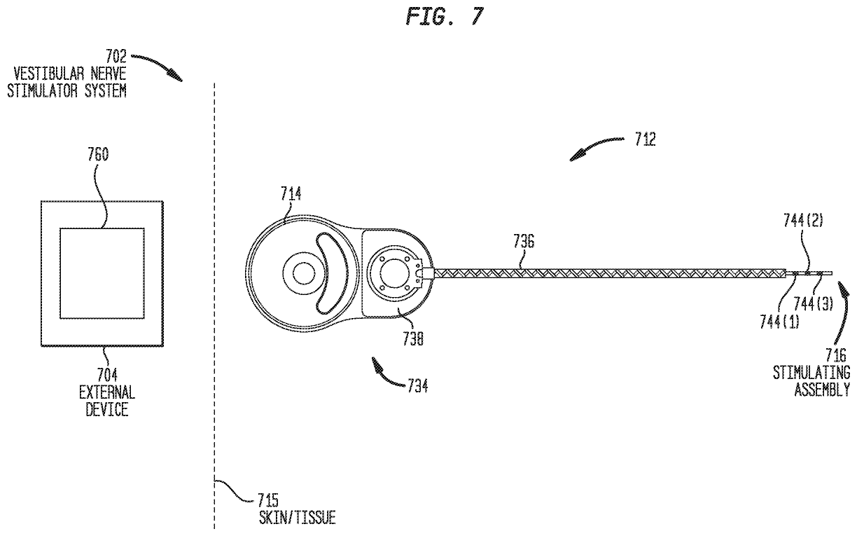

As previously described, the technology disclosed herein can be applied in any of a variety of circumstances and with a variety of different devices. Example devices that can benefit from technology disclosed herein are described in more detail in FIGS. 6 and 7 below. As described below, the operating parameters for the devices described with reference to FIGS. 6 and 7 can be configured according to the techniques described herein. The techniques of the present disclosure can be applied to other medical devices, such as neurostimulators, cardiac pacemakers, cardiac defibrillators, sleep apnea management stimulators, seizure therapy stimulators, tinnitus management stimulators, and vestibular stimulation devices, as well as other medical devices that deliver stimulation to tissue, to the extent that the operating parameters of such devices can be tailored based upon the posture of the user receiving the device. Further, technology described herein can also be applied to consumer devices. These different systems and devices can benefit from the technology described herein. For example, the operation techniques of the present disclosure can be applied to consumer grade or commercial grade headphone or ear bud products.

FIG. 6 is a functional block diagram of an implantable stimulator system 600 that can benefit from the technologies described herein. The implantable stimulator system 600 includes a wearable device 100 acting as an external processor device, and an implantable device 30 acting as an implanted stimulator device. In examples, the implantable device 30 is an implantable stimulator device configured to be implanted beneath a user's tissue (e.g., skin). In examples, the implantable device 30 includes a biocompatible implantable housing 602. Here, the wearable device 100 is configured to transcutaneously couple with the implantable device 30 via a wireless connection to provide additional functionality to the implantable device 30.

In the illustrated example, the wearable device 100 includes one or more sensors 612, a processor 614, a transceiver 618, and a power source 648. The one or more sensors 612 can be one or more units configured to produce data based on sensed activities. In an example where the stimulation system 600 is an auditory prosthesis system, the one or more sensors 612 include sound input sensors, such as a microphone, an electrical input for a frequency modulation (FM) hearing system, other components for receiving sound input, or combinations thereof. Where the stimulation system 600 is a visual prosthesis system, the one or more sensors 612 can include one or more cameras or other visual sensors. Where the stimulation system 600 is a cardiac stimulator, the one or more sensors 612 can include cardiac monitors. The processor 614 can be a component (e.g., a central processing unit) configured to control stimulation provided by the implantable device 30. The stimulation can be controlled based on data from the one or more sensors 612, a stimulation schedule, or other data. Where the stimulation system 600 is an auditory prosthesis, the processor 614 can be configured to convert sound signals received from the sensor(s) 612 (e.g., acting as a sound input unit) into signals 651. The transceiver 618 is configured to send the signals 651 in the form of power signals, data signals, combinations thereof (e.g., by interleaving the signals), or other signals. The transceiver 618 can also be configured to receive power or data. Stimulation signals can be generated by the processor 614 and transmitted, using the transceiver 618, to the implantable device 30 for use in providing stimulation.

In the illustrated example, the implantable device 30 includes a transceiver 618, a power source 648, and a medical instrument 611 that includes an electronics module 610 and a stimulator assembly 630. The implantable device 30 further includes a hermetically sealed, biocompatible implantable housing 602 enclosing one or more of the components.

The electronics module 610 can include one or more other components to provide medical device functionality. In many examples, the electronics module 610 includes one or more components for receiving a signal 651 and converting the signal 651 into a stimulation signal 615. The electronics module 610 can further include a stimulator unit. The electronics module 610 can generate or control delivery of the stimulation signals 615 to the stimulator assembly 630. In examples, the electronics module 610 includes one or more processors (e.g., central processing units or microcontrollers) coupled to memory components (e.g., flash memory) storing instructions that when executed cause performance of an operation. In examples, the electronics module 610 generates and monitors parameters associated with generating and delivering the stimulus (e.g., output voltage, output current, or line impedance). In examples, the electronics module 610 generates a telemetry signal (e.g., a data signal) that includes telemetry data. The electronics module 610 can send the telemetry signal to the wearable device 100 or store the telemetry signal in memory for later use or retrieval.

The stimulator assembly 630 can be a component configured to provide stimulation to target tissue. In the illustrated example, the stimulator assembly 630 is an electrode assembly that includes an array of electrode contacts disposed on a lead. The lead can be disposed proximate tissue to be stimulated. Where the system 600 is a cochlear implant system, the stimulator assembly 630 can be inserted into the user's cochlea. The stimulator assembly 630 can be configured to deliver stimulation signals 615 (e.g., electrical stimulation signals) generated by the electronics module 610 to the cochlea to cause the user to experience a hearing percept. In other examples, the stimulator assembly 630 is a vibratory actuator disposed inside or outside of a housing of the implantable device 30 and configured to generate vibrations. The vibratory actuator receives the stimulation signals 615 and, based thereon, generates a mechanical output force in the form of vibrations. The actuator can deliver the vibrations to the skull of the user in a manner that produces motion or vibration of the user's skull, thereby causing a hearing percept by activating the hair cells in the user's cochlea via cochlea fluid motion.

The transceivers 618 can be components configured to transcutaneously receive and/or transmit a signal 651 (e.g., a power signal and/or a data signal). The transceiver 618 can be a collection of one or more components that form part of a transcutaneous energy or data transfer system to transfer the signal 651 between the wearable device 100 and the implantable device 30. Various types of signal transfer, such as electromagnetic, capacitive, and inductive transfer, can be used to usably receive or transmit the signal 651. The transceiver 618 can include or be electrically connected to a coil 20.

As illustrated, the wearable device 100 includes a coil 108 for transcutaneous transfer of signals with the coil 20. As noted above, the transcutaneous transfer of signals between the coil 108 and the coil 20 can include the transfer of power and/or data from the coil 108 to the coil 20 and/or the transfer of data from the coil 20 to the coil 108. The power source 648 can be one or more components configured to provide operational power to other components. The power source 648 can be or include one or more rechargeable batteries. Power for the batteries can be received from a source and stored in the battery. The power can then be distributed to the other components as needed for operation.

As should be appreciated, while particular components are described in conjunction with FIG. 6, technology disclosed herein can be applied in any of a variety of circumstances. The above discussion is not meant to suggest that the disclosed techniques are only suitable for implementation within systems akin to that illustrated in and described with respect to FIG. 6. In general, additional configurations can be used to practice the methods and systems herein and/or some aspects described can be excluded without departing from the methods and systems disclosed herein.

FIG. 7 illustrates an example vestibular nerve stimulator system 702, with which embodiments presented herein can be implemented. As shown, the vestibular nerve stimulator system 702 comprises an implantable component (vestibular stimulator) 712 and an external device/component 704 (e.g., external processing device, battery charger, remote control, etc.). The external device 704 comprises a transceiver unit 760. As such, the external device 704 is configured to transfer data (and potentially power) to the vestibular stimulator 712. External device 704 can also include an inertial measurement unit analogous to inertial measurement unit 170 of FIG. 1D.

The vestibular stimulator 712 comprises an implant body (main module) 734, a lead region 736, and a stimulating assembly 716, all configured to be implanted under the skin (tissue) 715 of the user. The implant body 734 generally comprises a hermetically-sealed housing 738 in which RF interface circuitry, one or more rechargeable batteries, one or more processors, and a stimulator unit are disposed. The implant body 134 also includes an internal/implantable coil 714 that is generally external to the housing 738, but which is connected to the transceiver via a hermetic feedthrough (not shown). Implant body 734 can also include an inertial measurement unit analogous to inertial measurement unit 180 of FIG. 1D.

The stimulating assembly 716 comprises a plurality of electrodes 744(1)-(3) disposed in a carrier member (e.g., a flexible silicone body). In this specific example, the stimulating assembly 716 comprises three (3) stimulation electrodes, referred to as stimulation electrodes 744(1), 744(2), and 744(3). The stimulation electrodes 744(1), 744(2), and 744(3) function as an electrical interface for delivery of electrical stimulation signals to the user's vestibular system.

The stimulating assembly 716 is configured such that a surgeon can implant the stimulating assembly adjacent the user's otolith organs via, for example, the user's oval window. It is to be appreciated that this specific embodiment with three stimulation electrodes is merely illustrative and that the techniques presented herein can be used with stimulating assemblies having different numbers of stimulation electrodes, stimulating assemblies having different lengths, etc.

In operation, the vestibular stimulator 712, the external device 704, and/or another external device, can be configured to implement the techniques presented herein. That is, the vestibular stimulator 712, possibly in combination with the external device 704 and/or another external device, can include an evoked biological response analysis system, as described elsewhere herein.

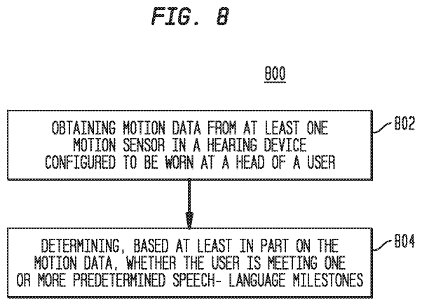

FIG. 8 is a flowchart of an example method 800, in accordance with embodiments presented herein. Method 800 begins at 802 where a system/device obtains motion data from at least one motion sensor in a hearing device configured to be worn at a head of a user. At 804, the system/device determines, based at least in part on the motion data, whether the user is meeting one or more predetermined speech-language milestones.

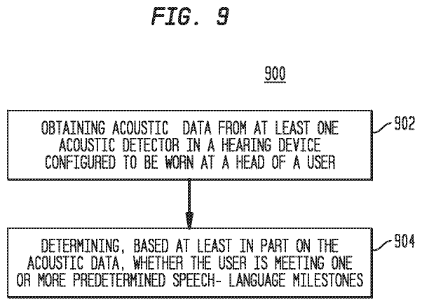

FIG. 9 is a flowchart of an example method 900, in accordance with embodiments presented herein. Method 900 begins at 902 where a system/device obtains acoustic data from at least one acoustic detector in a user device associated with a user. At 904, the system/device determines, based at least in part on the acoustic data, whether the user is meeting one or more predetermined speech-language milestones.

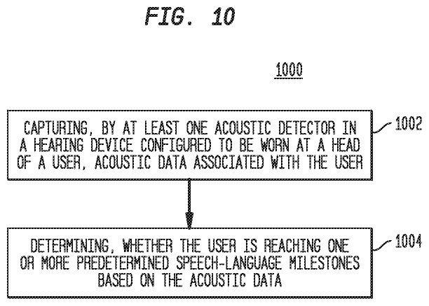

FIG. 10 is a flowchart of an example method 1000, in accordance with embodiments presented herein. Method 1000 begins at 1002 where at least one acoustic detector in a hearing device configured to be worn at a head of a user captures acoustic data associated with the user. At 1004, a determination is made as to whether the user is reaching one or more predetermined speech-language milestones based on the acoustic data.

### Additional Variations and Alternatives

As should be appreciated, while particular uses of the present technology have been illustrated and discussed above with reference to the accompanying drawings in which some of the possible aspects were shown, the disclosed technology can be used with a variety of devices in accordance with many examples of the technology. The above discussion is not meant to suggest that the disclosed technology is only suitable for implementation within systems akin to that illustrated in the figures. As should be appreciated, the various aspects (e.g., portions, components, etc.) described with respect to the figures herein are not intended to limit the systems and processes to the particular aspects described. In general, additional configurations can be used to practice the processes and systems herein and/or some aspects described can be excluded without departing from the processes and systems disclosed herein. Other possible aspects can be embodied in many different forms and should not be construed as limited to the aspects set forth herein. Rather, these aspects were provided so that this disclosure was thorough and complete and fully conveyed the scope of the possible aspects to those skilled in the art.

According to certain aspects, systems and non-transitory computer readable storage media are provided. The systems are configured with hardware configured to execute operations analogous to the methods of the present disclosure. The one or more non-transitory computer readable storage media comprise instructions that, when executed by one or more processors, cause the one or more processors to execute operations analogous to the methods of the present disclosure.

Similarly, where steps of a process are disclosed, those steps are described for purposes of illustrating the present methods and systems and are not intended to limit the disclosure to a particular sequence of steps. For example, the steps can be performed in differing order, two or more steps can be performed concurrently, additional steps can be performed, and disclosed steps can be excluded without departing from the present disclosure. Further, the disclosed processes can be repeated.

Although specific aspects were described herein, the scope of the technology is not limited to those specific aspects. One skilled in the art will recognize other aspects or improvements that are within the scope of the present technology. Therefore, the specific structure, acts, or media are disclosed only as illustrative aspects. The scope of the technology is defined by the following claims and any equivalents therein.

It is also to be appreciated that the embodiments presented herein are not mutually exclusive and that the various embodiments can be combined with another in any of a number of different manners and arrangements.

The invention described and claimed herein is not to be limited in scope by the specific preferred embodiments herein disclosed, since these embodiments are intended as illustrations, and not limitations, of several aspects of the invention. Any equivalent embodiments are intended to be within the scope of this invention. Indeed, various modifications of the invention in addition to those shown and described herein will become apparent to those skilled in the art from the foregoing description. Such modifications are also intended to fall within the scope of the appended claims.

1. A method comprising:
obtaining motion data from at least one motion sensor in a device configured to be worn at a head of a user; and
determining, based at least in part on the motion data, whether the user is meeting one or more predetermined speech-language milestones.

2. The method of claim 1, wherein the motion data is data indicative of attentional focus of the user.

3. The method of claim 2, wherein the motion data is one or more of electrooculography (EOG) sensor data, electromyography (EMG) sensor data, and/or inertial measurement unit (IMU) sensor data for tracking eye, mouth, head and/or body movements of the user.

4. The method of claim 1, wherein the user is a pediatric user and the one or more predetermined speech-language milestones include one or more pediatric speech-language developmental milestones.

5. The method of claim 4, wherein determining whether the user is meeting the one or more predetermined speech-language milestones comprises:
comparing the motion data to age-specific normative values for respective pediatric speech-language developmental milestones; and
determining whether the motion data is consistent with the age-specific normative values in connection with at least one of the pediatric speech-language developmental milestones.

6. The method of claim 5, further comprising outputting an indication that the pediatric user is on-track when the motion data is consistent with the age-specific normative values in connection with one of the pediatric speech-language developmental milestones.

7. The method of claim 5, further comprising outputting an indication that the pediatric user is delayed or at risk of developmental delay when the motion data is inconsistent with the age-specific normative values in connection with one of the pediatric speech-language developmental milestones.

8. The method of claim 1, further comprising:
obtaining acoustic data from at least one acoustic detector in the device; and
determining, based on the motion data in further combination with the acoustic data, whether the user is meeting the one or more predetermined speech-language milestones.

9. The method of claim 8, further comprising:
performing an own voice detection (OVD) process on the acoustic data to distinguish vocal input of the user from vocal input of others and/or background noise in a surrounding environment of the user, and generate OVD information based on the vocal input of the user; and
determining whether the user is meeting the one or more predetermined speech-language milestones based on the OVD information associated with the vocal input of the user.

10. The method of claim 9, wherein determining whether the user is meeting the one or more predetermined speech-language milestones comprises calculating one or more speech-language patterns for assessing speech-language development of the user based on the OVD information associated with the vocal input of the user.

11. The method of claim 8, wherein the acoustic data further includes non-OVD linguistic input captured via a microphone included in the device.

12. The method of 1, further comprising:
receiving user information associated with the user, wherein the user information includes one or more of an age of the user and an indication of a developmental delay associated with the user; and
determining whether the user is meeting the one or more predetermined speech-language milestones based on the user information.

13. The method of claim 1, further comprising logging whether the user is meeting the one or more predetermined speech-language milestones in a log with the motion data.

14-34. (canceled)

35. A device, comprising:
a network adapter for communication with a user device associated with a user, where the user device includes one or more motion sensors and one or more acoustic detectors;
memory; and
one or more processors, wherein the one or more processors are configured to:
obtain motion data associated with the user from the one or more motion sensors,
obtain acoustic data from the one or more acoustic detectors,
determine whether the user of the user device is meeting one or more predetermined speech-language milestones based on at least one of the motion data or the acoustic data, and
one or more output devices configured to provide an indication of whether the user of the user device is meeting the one or more predetermined speech-language milestones.

36. (canceled)

37. The device of claim 35, wherein the one or more predetermined speech-language milestones include one or more pediatric speech-language developmental milestones, and wherein, in determining whether the user is meeting the one or more predetermined speech-language milestones, the one or more processors are configured to:
compare the motion data or the acoustic data or a combination thereof to age-specific normative values for respective pediatric speech-language developmental milestones; and determine whether the motion data or the acoustic data or a combination thereof is
consistent with the age-specific normative values in connection with at least one of the pediatric speech-language developmental milestones.

38. The device of claim 37, wherein the one or more processors are further configured to output an indication that the user is on-track when the motion data or the acoustic data or the combination thereof is consistent with the age-specific normative values in connection with one of the pediatric speech-language developmental milestones.

39. The device of claim 37, wherein the one or more processors are further configured to output an indication that the user is delayed or at risk of developmental delay when the motion data or the acoustic data or the combination thereof is inconsistent with the age-specific normative values in connection with one of the pediatric speech-language developmental milestones.

40. The device of claim 35, or wherein the one or more predetermined speech-language milestones are associated with cognitive decline, and wherein, in determining whether the user is reaching the one or more predetermined speech-language milestones, the one or more processors are further configured to:
compare the acoustic data to user-specific baseline values and/or historical values associated with respective speech-language patterns for assessing speech-language abilities of the user; and
identify that the user is experiencing cognitive decline over time when a result of the comparing indicates a change of at least a threshold value from the user-specific baseline values and/or historical values in connection with one of the speech-language patterns.

41. The device of claim 35, or wherein the one or more predetermined speech-language milestones are associated with speech production decline, and wherein, in determining whether the user is reaching the one or more predetermined speech-language milestones, the one or more processors are further configured to:
compare the acoustic data to user-specific baseline values and/or historical values associated with amplitude, phonological errors, rate of speech, or combinations thereof; and
identify that the user is experiencing speech production decline over time when a result of the comparing indicates decreasing amplitude, increasing phonological errors, decreasing rate of speech, or combinations thereof.

42. (canceled)

43. (canceled)

44. The device of claim 35, or wherein the one or more processors are further configured to:
log whether the user is reaching the one or more predetermined speech-language milestones in a log with the motion data or the acoustic data or a combination thereof; and
predict a future speech-language milestone for the user based on information stored in the log over time.

45. (canceled)
